(19) United States
(12) Patent Application Publication
(10) Pub. No.: US 2005/0171484 A1
(43) Pub. Date: Aug. 4, 2005

(54) Syringe needle de-capping and re-capping device

(76) Inventor: Michael J. Jangula, Lynnwood, WA (US)

Correspondence Address: DEAN A. CRAINE, P.S.;SUITE 140, 400-112TH AVE. NE, BELLEVUE, WA, 98004, US

(21) Appl. No.: 10/770,255

(22) Filed: Feb. 2, 2004

(51) Int. Cl. A61M 5/32

(52) U.S. Cl. 604/198

(57) ABSTRACT

A syringe needle de-capping and re-capping device that includes a cylindrical body with a finger gripping member integrally formed thereon. Formed inside the body is a cylindrical-shaped cavity designed to receive a bushing. Formed along the upper edge of the cavity are internal threads that connect to external threads located on a removable cap that removeably attaches the cap to the body. Attached to the cap is a neck that extends into the bushing. Formed inside the bushing is a center cavity with a spring nut disposed therein designed to receive and partially engage the tip of the needle cap when inserted through the cap and into the cavity. When properly assembled, the neck on the cap holds the spring nut inside the bushing. During use, the device is held between the user's fingers with the body extending above the user's fingers so that the user may used his or her palm to hold the vial. The opposite hand holds the syringe so that needle cap may be easily de-cap and re-cap the needle.

### BACKGROUND OF THE INVENTION

1. Field of the Invention

The invention relates to hypodermic syringes, and more particularly to devices used to protect the user's fingers from accidental needle sticks and excessive radiation exposure when handling syringes containing radioactive medications or fluids.

2. Description of the Related Art

It is common to administer radioactive medications or fluids to patients. In many regions in the United States, syringes filled with radioactive medications or fluids are filled in advance by a trained pharmacist or technician in special facilities, such as nuclear pharmacies. Often the task of filling syringes with a radioactive medication or fluid is assigned to one person who prepares at one time a large number of syringes for different departments or healthcare facilities in the region. Because large numbers of syringes are prepared, needle sticks are common and the amount of radiation exposure to the pharmacist or technician may exceed safety levels.

When preparing syringes filled with radioactive medications or fluids, the pharmacist or technician must use sterile techniques. Typically, a large volume of radioactive medication or fluid is prepared in advance and delivered to a laminar flow, mixing hood. The pharmacist or technician then manually selects a labeled, sterile syringe, removes the protective cap covering the needle and inserts the tip of the needle into the rubber seal on the vial to withdraw a desired amount of radioactive medication or fluid. After the desired amount of radioactive medication or fluid is withdrawn, the needle is withdrawn from the vial and the protective cap is placed back over the needle. The filled syringe is then placed to one side and an empty syringe is then selected and filled in the same manner. When all of the syringes are filled, they are then placed in packaging for delivery to the department or facility where the medication will be administered.

In order to keep the needle of a syringe sterile and to prevent accident needle sticks, the protective needle cap is carefully removed from the needle referred as de-capping, to fill the syringe and then carefully placed over the needle referred as re-capping, when the syringe is filled. An important skill mastered by the pharmacist and technician is sequentially moving the hands and fingers to simultaneously hold the syringe, remove the protective cap, securely hold the large vial of radioactive medication or fluid at a suitable angle to withdraw fluid from the vial, insert the tip of the needle into the top seal or gasket on the vial at a suitable angle to prevent “coring”, manipulate the syringe to withdraw the desired amount of radioactive medication or fluid from the vial, withdraw the needle from the vial, and then re-cap the needle. All of these acts must be accomplished repeatedly without accidentally sticking the pharmacist or technician with the needle or excessively exposing the pharmacists or technician's hands and fingers to radiation. Unfortunately, accidental needle sticks and excessive radiation exposure are common.

What is needed is a device that enables a user to quickly and easily fill single or multiple syringes with a desired medication or fluid using a sterile technique that protects against accidental needle sticks and protects the user's fingers and hands from excessive radiation.

### SUMMARY OF THE INVENTION

It is an object of the present invention to provide a device that protects the user's fingers and hands from needle sticks when de-capping and re-capping a syringe filled with a medication or fluid.

It is another object of the present invention to provide a device used to protect the user's fingers and hands from radiation exposure when withdrawing radioactive medications and fluids into a syringe.

It is a further object of the present invention to provide such a device that allows the user to hold the needle cap and vial in the same hand so that the other hand can easily manipulate the syringe.

These and other objects of the invention are met by a protective syringe needle de-capping and re-capping device that includes a cylindrical-shaped body with a longitudinally aligned bushing cavity formed therein. Formed around the outer edge of the cavity are internal threads that connect to external threads located on a removable cap. Perpendicularly aligned on the cap is an inward extending neck. Extending through the neck and cap is a longitudinally aligned needle cap passageway. In the preferred embodiment, the neck is an adapter separately attached to the cap.

Located inside the bushing cavity is a longitudinally aligned bushing. The bushing includes a cylindrical-shaped, longitudinally aligned void area designed to receive the neck on the adapter. Located inside the void area is a spring nut with a center bore designed to receive and connect to the tip of a needle cap. A recessed edge is formed inside the bushing, which acts as a stop surface for the spring nut when in a fixed position inside the bushing.

During assembly, the spring nut is inserted into the bushing. The neck on the adapter is then longitudinally aligned and extended into the bushing and against the spring nut. The removable cap is then attached to the adapter and to the body. A needle cap on a syringe may be inserted into the needle cap passageway and rotated in a clockwise direction. As the needle is rotated, the tip of the needle cap engages the spring nut so that the needle cap disconnects from the hub on the syringe when the syringe is pulled outward. The needle cap remains connected to the spring nut. Later, the needle is re-inserted into the needle cap passageway and pressed inward and rotated in a counter-clockwise direction to reconnect the needle cap to the syringe.

The body includes a finger gripping section that enables the device to be easily held in one hand. In a first embodiment, the finger gripping section is perpendicularly aligned with the body so that when the finger gripping section is held between two fingers, the body is aligned substantially perpendicular to the top surface of the hand. In the second embodiment, the finger gripping section is longitudinally aligned on one end of the body so that when the finger gripping section extends between the user's two fingers, the body is disposed above and substantially parallel to the top surface of the hand. In a third embodiment, the finger-gripping surface is a conical surface formed on the body.

Optionally, the bushing is made of radiation shielding material, such as lead, which reduces the amount of radiation exposure to the user's fingers and hands.

### DESCRIPTION OF THE DRAWINGS

FIG. 1 is an illustration of the first embodiment of a syringe needle de-capping and re-capping device disclosed herein shown held between the index and middle fingers on one hand also holding a vial filled with radioactive medication or fluid while the other hand holds and operates a syringe used to withdraw radioactive medication or fluid from the vial.

FIG. 2 is an exploded, perspective view of the device shown in FIG. 1 showing the proper alignment of the syringe with respect to the small opening on the removable cap.

FIG. 3 is a perspective view of the device shown in FIG. 2 with a syringe being inserted into the assembled device.

FIG. 4 is a sectional, left side elevational view of the syringe and device shown in FIG. 3.

FIG. 5 is a front elevational view of the device with the removable cap removed.

FIG. 6 is a sectional, side elevational view of a second embodiment of the device.

FIG. 7 is a sectional, side elevational view of a third embodiment of the device.

### DESCRIPTION OF THE PREFERRED EMBODIMENT(S)

There is shown in the accompanying Figs. three embodiments of a needle syringe de-capping and re-capping device, generally referenced as 10, 10′, 10″ specifically designed for drawing medication 8 or fluid from a vial 7 held by one hand into a standard syringe 90 held by the other hand (not shown).

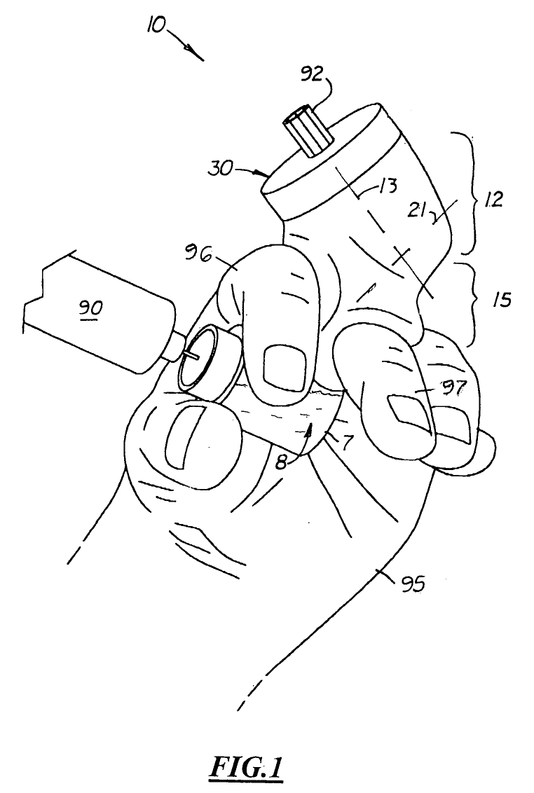

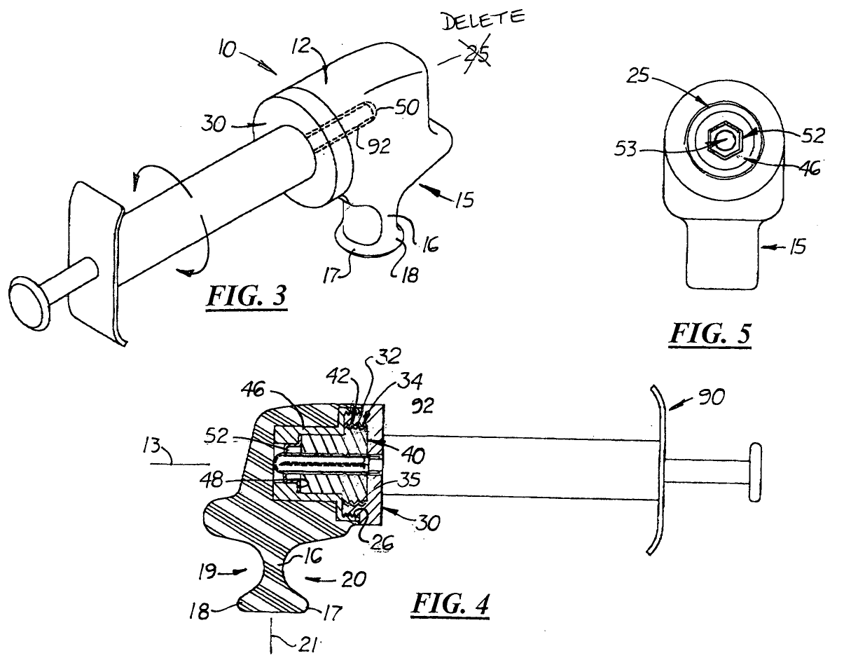

The first embodiment of the device, shown in FIGS. 1-5 and denoted 10, includes a cylindrical—shaped body 12 with a lower, substantially perpendicular t-shaped finger gripping section 15 integrally formed on the body 12 that enables the device 10 to be held in one hand 95 between two fingers 96, 97. The finger gripping section 15 includes a middle leg 16 and two laterally extending wings 17, 18. Finger openings 19, 20 are formed between the sides of the middle leg 16 and the wings 17, 18 designed to receive the index and middle fingers 96, 97 respectively.

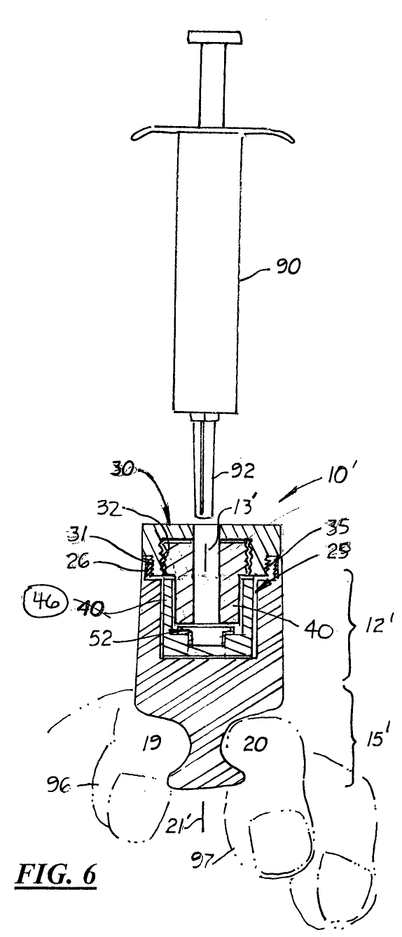

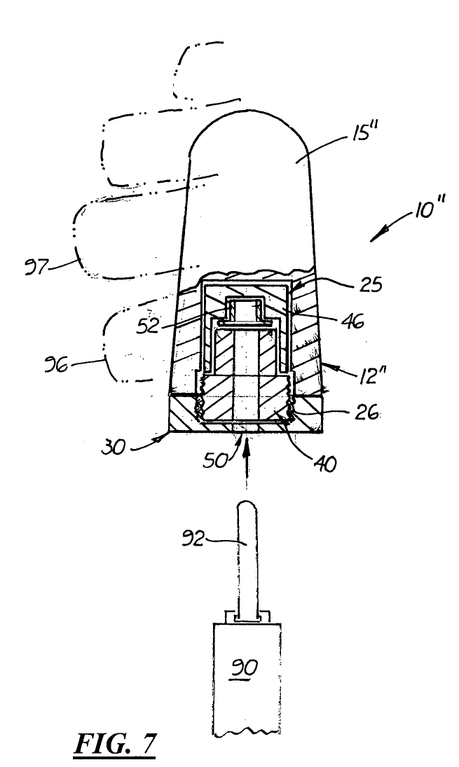

The longitudinal axis 13 of the body 12 is a substantially perpendicular (75°-115°) angle aligned with the center longitudinal axis 21 of the finger gripping section 15. The body 12 is sufficient in length and diameter so that its longitudinal axis 13 is disposed approximately (2.0-2.5) cm above the top surface of the fingers 96, 97 when held. The center axis of the finger openings 19, 20 are slightly offset so that the longitudinal axis 13 of the body 12 is diagonally aligned over the top surface of fingers 96, 97 when the device 10 is held. In a second embodiment of the device, denoted 10′ and shown in FIG. 6, the longitudinal axis 13′ of the body 12′ is longitudinally aligned with the longitudinal axis 21′ of the lower finger gripping surface 15′. In a third embodiment of the device, denoted 10″, the body 12″ includes a conical-shaped finger gripping section 15″ that enables the device 10″ to be held transversely in the fingers and palm of the hand.

Formed inside the body 12, 12′, 12″, with each device 10, 10′, 10″, respectively, is a longitudinally aligned cylindrical-shaped bushing cavity 25. Formed on the inside surface near the outer edge of the bushing cavity 25 are internal threads 26. During assembly, the threads 26 connect to external threads 32 formed on the removable cap 30.

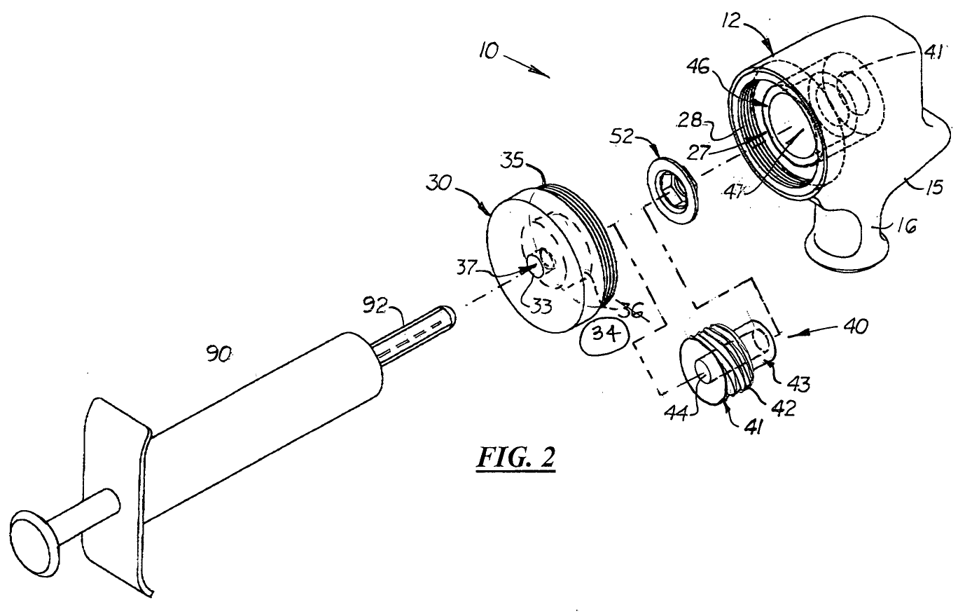

In FIGS. 2 and 4, a separate adaptor 40 attaches to removable cap 30 and extends inward to the bushing cavity 25. The adaptor 40 includes a cylindrical-shaped, large diameter main section 41 and an integrally formed, cylindrical-shaped, small diameter, non-threaded neck section 43. The main section 41 includes external threads 42 that selectively connect to the internal threads 35 formed on a threaded adapter, receiving cavity 34 formed on the removable cap 30. During the assembly, the adapter 40 is attached to the removable cap 30 and the neck section 43 is longitudinally aligned and inserted into the void area 47 formed inside a bushing 46. The length of the neck section 43 is sufficient to press against a spring nut 52 located inside the bushing 46. Formed centrally on the removable cap 30 and the adapter 40 are first and second small openings 33, 44, respectively, that form a needle cap passageway 50 designed to receive a standard needle cap 92.

The bushing 46 is aligned longitudinally inside the bushing cavity 25. As shown more clearly in FIG. 4, formed inside the bushing 46 is an inward extending stop surface 48 upon which the spring nut 52 is disposed. When the removable cap 30 is attached to the body 12, the neck section 43 extends into the cavity 47 to hold the spring nut 52 against the stop surface 48. When the needle cap 92 is inserted into the needle cap passageway 50, the tip of the needle cap 92 is inserted into the center bore 53 on the spring nut 52. When the syringe 90 is rotated in a clockwise direction, the spring nut 52 engages the tip of the needle cap 92 to de-cap the syringe 90. When the syringe 90 is pulled outward, the needle cap 92 remains connected to the device 10. Later, when the needle is re-inserted into the needle cap passageway 50, pressed inward and rotated in a counter-clockwise direction, the needle cap 92 reconnects to the syringe 90.

Each embodiment of the device 10, 10′, 10″ is designed to be use with a bushing 46 that aligns and holds the spring nut 52 inside the bushing cavity 25. In the preferred embodiment, the bushing 46 is made of radiation shielding material, such as lead, to reduce radiation exposure to the user's fingers 96, 97. It should be understood, however, that the bushing 46 may be made of non-radiation shielding material, e.g. aluminum, which may be used when not handling radioactive medications or fluids.

In the preferred embodiment, the cylindrical body 12 is made of molded plastic molded rubber or aluminum and measures approximately 7 cm in height, 5.4 cm in width, and 4.2 cm in depth. The small opening 33 on the removable cap 30 measures approximately 0.7 cm in diameter. In the preferred embodiment, the bushing 46 is approximately 2.4 cm length and 2.2 cm in diameter. The sidewalls of the bushing 46 are approximately 0.3 cm thick. The adapter 40 is approximately 2 cm in length with the wide section 41 of the adapter 40 being approximately 2 cm in diameter. The neck section 43 of the adapter 40 is approximately 1.5 cm in length and 1.75 cm in diameter.

In compliance with the statute, the invention described herein has been described in language more or less specific as to structural features. It should be understood, however, that the invention is not limited to the specific features shown, since the means and construction shown is comprised only of the preferred embodiments for putting the invention into effect. The invention is therefore claimed in any of its forms or modifications within the legitimate and valid scope of the amended claims, appropriately interpreted in accordance with the doctrine of equivalents.

1. A syringe needle de-capping and re-capping device, comprising:
a. a body with longitudinally aligned cavity formed therein, said body including a finger gripping section;
b. a removable cap selectively attachable to said body, said cap including an inward extending aligned neck;
c. a bushing longitudinally aligned and located inside said cavity, said bushing including a cylindrical shaped void area capable of receiving said neck on said removable cap, said bushing includes a stop surface formed inside said void area, and;
d. a spring nut located inside said void area of said bushing, said spring nut including a center bore that engages the tip of a needle cap with inserted therein.

2. The syringe needle de-capping and re-capping device, as recited in claim 1, wherein said finger gripping member that allows said body to be held between a user's finger so that said cavity is located above the top surface of the user's fingers.

3. The syringe needle de-capping and re-capping device as recited in claim 2, wherein said body and said finger gripping member are longitudinally aligned so that when a user's fingers engage said finger gripping member, said cylindrical body extends upward substantially perpendicular to the top surface of the fingers used to hold said device.

4. The syringe needle de-capping and re-capping device, as recited in claim 1, wherein said bushing is made of radiation shielding material.

5. The syringe needle de-capping and re-capping device, as recited in claim 2, wherein said bushing is made of radiation shielding material.

6. The syringe needle de-capping and re-capping, as recited in claim 3, wherein said bushing is made of radiation shielding material.

7. The syringe needle de-capping and re-capping device as recited in claim 1, wherein said body and said finger gripping member are made of molded rubber.

8. The syringe needle de-capping and re-capping device as recited in claim 1, further including internal threads formed on said cavity and said cap including external threads that selectively interconnect to attached said cap to said body.

9. The syringe needle de-capping and re-capping device as recited in claim 1, wherein said neck is an adaptor removably attached to said cap.

10. The syringe needle de-capping and re-capping device, as recited in claim 9, further including a bushing made of radiation shielding material.

11. The syringe needle de-capping and re-capping device as recited in claim 1, wherein said body and said finger gripping member are perpendicularly aligned so that when a user's fingers engage said finger gripping member, said cylindrical body extends transversely over the top surface of the user's fingers.

12. The syringe needle de-capping and re-capping device, as recited in claim 11, wherein said bushing is made of radiation shielding material.

13. The syringe needle de-capping and re-capping device, as recited in claim 1, when said finger gripping member is conical.

14. A syringe needle de-capping and re-capping device, comprising:
a. a cylindrical body with longitudinally aligned bushing cavity formed therein;
b. a t-shaped finger griping member longitudinally aligned and formed on said body capable of being engaged between two fingers on a user's hand;
c. a removable cap attached to said cylindrical body to selectively close said cavity, said removable cap including a small opening capable of receiving a needle cap;
d. a bushing located inside said cavity formed in said body, said bushing including a void area; and,
e. a spring nut located inside said void area of said bushing capable to engaging the tip of a needle cap when inserted through said small opening on said removable cap and into said void area in said bushing.

15. The syringe needle de-capping and re-capping device, as recited in claim 14, wherein said bushing is made of a radiation shielding material.

16. The syringe needle de-capping and re-capping device as recited in claim 14, further including an adapter attached to said cap that extends into said bushing to hold said spring nut inside said bushing.

17. The syringe needle de-capping and re-capping device, as recited in claim 16, wherein said bushing is made of radiation shielding material.

18. A syringe needle de-capping and re-capping device, comprising:
a. a cylindrical body with longitudinally aligned cavity formed therein;
b. a t-shaped finger gripping member located on one side of said body capable of being engaged between two fingers on a user's hand;
c. a removable cap attached to said body to selectively close said cavity, said removable cap including a small opening capable of receiving a needle cap;
d. a bushing located inside said cavity formed in said body said bushing including a void area; and,
e. a spring nut located inside said void area of said bushing capable to engaging the tip of a needle cap when inserted through said small opening on said removable cap and into said void area in said bushing.

19. The syringe needle de-capping and re-capping device, as recited in claim 18, wherein said bushing is made of radiation material.

20. The syringe needle de-capping and re-capping device, as recited in claim 18, wherein said bushing is made of radiation shielding material.
